(19) United States
(12) Patent Application Publication
(10) Pub. No.: US 2023/0064307 A1
(43) Pub. Date: Mar. 2, 2023

(54) Pharmaceutical composition to destabilize abnormal methylation enzymes and use thereof

(71) Applicants: Ming C. Liau, Missouri City, TX (US); Christine Liau Craig, Houston, TX (US); Linda Liau Baker, Missouri City, TX (US); John P. Fruehauf, Tustin, CA (US)

(72) Inventors: Ming C. Liau, Missouri City, TX (US); Christine Liau Craig, Houston, TX (US); Linda Liau Baker, Missouri City, TX (US); John P. Fruehauf, Tustin, CA (US)

(21) Appl. No.: 17/300,593

(22) Filed: Aug. 28, 2021

(51) Int. Cl. A61K 31/57; A61K 31/198; A61K 31/202; A61K 31/196; A61K 31/12

(52) U.S. Cl. CPC A61K 31/57; A61K 31/12; A61K 31/196; A61K 31/198; A61K 31/202

(57) ABSTRACT

The present invention relates to the discoveries of the mechanism of wound healing, the relationship of wound healing to the evolution of cancer, and the use of such discoveries for the perfection of wound healing to avoid ugly scar and cancer evolution, and for the use to take out both cancer cells and cancer stem cells to accomplish a permanent cure of cancer.

### BACKGROUND OF THE INVENTION

### Field of the Invention

The invention relates generally to pharmaceutical compositions to destabilize abnormal methylation enzymes for the perfection of wound healing to avoid ugly scar and cancer evolution, and more particularly, for the use to take out both cancer cells and cancer stem cells to accomplish a permanent cure of cancer thereof.

### Description of the Related Art

### Chemo-Surveillance (Chemo-Sur) as a Natural Mechanism for the Perfection of Wound Healing

Chemo-sur was a hypothesis brought up by Liau et al. as a natural defense mechanism against cancer [1]. This hypothesis was based on the observation that healthy people were able to maintain a steady level of metabolites active as differentiation inducers (Dls) and differentiation helper inducers (DHIs), whereas cancer patients tended to show deficiencies of such metabolites due to cachexia symptom causing excessive urinary excretion of such metabolites. Dls and DHIs are metabolites that can modulate differentiation capability of cells with abnormal methylation enzymes (MEs). MEs of cancer cells are abnormal due to association with telomerase [2].

The association of MEs with telomerase locks MEs in an exceptionally stable and active state to block terminal differentiation (TD) of cancer cells [3, 4]. Dls are chemicals capable of eliminating telomerase from abnormal MEs, and DHls are inhibitors of the ternary MEs consisting of methionine adenosyltransferase (MAT)-methyltransferase (MT)-S-adenosylhomocysteine hydrolase (SAHH) [5]. It turns out that Chemo-sur is in fact a natural mechanism to ensure wound healing, because the induction of TD of progenitor stem cells (PSCs) is a critical mechanism for the perfection of wound healing [6, 7]. MEs of PSCs are abnormal like cancer cells. Mechanism on the induction of TD operating on cancer cells is applicable to PSCs.

Wound healing requires the proliferation and the TD of PSCs. Since we do not know how to handle PSCs, we let the nature take its course to heal the wound. Wounds are always healed perfectly without having to put up any effort. Wound triggers biological response and immunological response. The biological response is good for the wound healing. But the immunological response is bad for the wound healing. Biological response involves the release of arachidonic acid (AA) from membrane bound phosphatidyl inositol by phospholipase A2 for the synthesis of prostaglandins (PGs) by cyclooxygenase and prostaglandin synthase [8, 9].

Production of PGs is essential for the efficacious wound healing. We have found that some PGs were very good Dls [10, 11]. Induction of TD, however, at the initial stage of wound is not their objective. Most PGs are unstable [8]. Their biological effect is very likely brief and confined to the wound area. PGs cause inflammation which may facilitate local membrane hyperpermeability for the extravasation of Dls and DHIs for the proliferation of PSCs to heal the wound [12]. Dls and DHIs normally function as a brake to inhibit the buildup of PSCs. The release of brake is very likely the important function of PGs at the initial stage of wound. PGs are probably no longer available at the final stage of wound healing which requires TD of PSCs. So the terminal stage of wound healing must be carried out by surveillance Dls and DHIs. Thus, the functionality of Chemo-sur becomes an important factor to dictate the success of wound healing [7, 13]. If the functionality of Chemo-sur is intact as healthy people, perfect wound healing can always be expected.

On the contrary, if the functionality of Chemo-sur has been damaged due to long term immuno- or toxic chemo-disorders, then TD of PSCs will be affected to result in scar, ugly scar, or worse evolution of cancer. Afterall, PSCs are normal stem cells which obey the rule of contact inhibition. That rule cannot restrict cancer stem cells (CSCs), which can pile up to make the scar ugly or even worse progress to cancer. Therefore, the integrity of Chemo-sur is so important for the perfection of wound healing to avoid ugly scar and to avoid cancer evolution.

### Cancer Arises as a Consequence of Wound Not Healing Properly

Wound healing and the evolution of cancer are closely related to involve PSCs as the critical common elements. It takes only a single hit by silencing ten-eleven translocation dioxygenase-1 (TET-1) enzyme to convert PSCs to cancer stem cells (CSCs), which is well within the reach of PSCs equipped with exceptionally active abnormal MEs. TET-1 enzyme is responsible for the lineage transition of PSCs, which is silenced in cancer cells [14-18]. Thus, differentiation programs including lineage transitions are completely shut down in cancer cells. The progression to activate oncogenes or to inactivate suppressor genes can turn CSCs to faster growing cancer cells to proliferate perpetually.

Acute wound affects Chemo-sur only temporarily, which is quickly restored to the normal state. The good effect of biological response to wound healing prevails in this case. It is the chronic wound that produces a persistent damage to the functionality of Chemo-sur to impair the ability to heal wound, resulting in the evolution to cancer. The bad effect of immunological response to wound healing prevails in this case. Myelodysplastic syndrome . (MDS) is a good example of cancer evolution due to wound not healing properly. MDS often starts with a display of an immunological disorder [19], which prompts the production of inflammatory cytokines. Among cytokines produced, tumor necrosis factor (TNF) is the critical factor related to the development of MDS [20]. It causes excessive apoptosis of bone marrow stem cells, thus severely affecting the ability of the patient to produce hematopoietic cells such as erythrocytes, platelets, and neutrophils. TNF is also named cachectin, because of its causation of cachexia symptom commonly shared by cancer and inflammatory patients. A characteristic disorder of cachexia is the excessive urinary excretion of low molecular weight metabolites, because of vascular hyperpermeability caused by TNF [21, 22].

As a consequence, Chemo-sur normally operating in healthy people to keep PSCs in check becomes dysfunctional, allowing PSCs to buildup in order to replenish unipotent stem cells wiped out by TNF. The high level of telomerase in the peripheral and bone marrow leukocytes is an indication of the widespread multiplication of PSCs [23, 24]. The multiplication of PSCs always runs a risk for PSCs to evolve into CSCs. During the course of MDS progression, mutations affecting enzymes were frequently observed [25-27], which might play significant roles in the evolution of PSCs to become CSCs [28]. As anemia in MDS patients becomes worse, chromosomal abnormalities such as translocations and deletions characteristic of cancer cells arise to accelerate replication, eventually pushing MDS patients to progress to acute myeloid leukemia (AML) [29-32]. The occurrence of AML is a classical example that cancer is originated from PSCs due to the collapse of the functionality of Chem-sur, allowing PSCs to evolve into CSCs, and then to progress to faster growing cancer AML.

Cancer due to wound not healing properly is not unique to AML. It is rather a common phenomenon. We have previously observed that the protection of the integrity of Chemo-sur by Antineoplaston A10, namely phenylacetylglutamine, could effectively prevent chemical carcinogenesis [33, 34], and achieve effective therapy of early stage cancer [1]. We have also observed that abnormal MEs were detectable in preneoplastic hyperplastic nodules before the appearance of carcinomas during chemical hepatocarcinogenesis [35]. This was an indication that carcinomas were derived from cells expressing abnormal MEs in the preneoplastic state, which were very likely PSCs. So the occurrence of human cancers and experimental animal cancers all points to PSCs’as the origin of cancers [36]

### Restoration of the Functionality of Chemo-Sur is Essential for the Success of Cancer Therapy

Evidently evolution of CSCs from PSCs is a very easy and simple matter. The nature creates Chemo-sur to prevent that from happening. Chemo-sur is a very important natural mechanism to ensure wound healing to avoid cancer. Whatever comes naturally we always credit it to the nature’s creation to benefit human being. Photo synthesis that turns CO₂ into O₂ is a prime example. Other such as immuno-surveillance against infectious diseases is well accepted. We must also accept Chemo-sur as the nature’s creation against devastating disease of cancer. Obviously leaky renal tubules triggered by TNF are responsible for the collapse of the functionality of Chemo-sur and the development of cancer. Restoration of the functionality of Chemo-sur becomes an important matter for the success of cancer therapy [37]. In this regard, phenylacetylglutamine may have an important role to play, which we have found effective to fix leaky renal tubules to restore the functionality of Chemo-sur [1].

### Wound Healing Metabolites as the Best Candidates to Take Out CSCs

CSCs are originated from PSCs. Naturally CSCs display cell features and biological missions very similar to PSCs. Both PSCs and CSCs express adenosine triphosphate (ATP) binding drug pumps that effectively exclude toxic chemicals, and have upregulated anti-apoptosis programs that negate the pro-apoptotic signals activated by DNA damaging therapies [38-41]. Thus, these cells are resistant to cytotoxic drugs and radiation. These cells normally reside in acidic and hypoxic microenvironments hard to reach by the blood stream. They remain dormant unless situations such as wounds arise that stimulate their recruitment. Although CSCs constitute only a small side population, they are the primary cause of treatment failure in the past based on destruction strategy [42-44]. The primary causes of treatment failure such as metastasis, drug resistance, angiogenesis, and recurrence can all attribute to CSCs. It is apparent that CSCs stand in the way to deny the success of destruction therapies to put cancer away in the past [6, 45, 46]. Therefore, the ability of the drug to eradicate CSCs becomes an important consideration for the evaluation of cancer drugs [47].

Monoclonal antibodies and interference RNA against the expression of telomerase were vigorous pursued in the past to take out CSCs. We are still waiting for such agents to display clinical efficacy. Since CSCs reside in microenvironments hard to reach by the blood stream, small molecules easily diffusible such as wound healing metabolites are a better choice. In fact such molecules are routinely employed by PSCs on wound healing. Afterall, wound healing metabolites are the partners of the biological missions of PSCs and CSCs; they are easily tolerated by these cells equipped with drug resistance mechanism. Cell differentiation agent-2 (CDA-2) was a preparation of wound healing metabolites purified from freshly collected human urine [48]. CDA-2 is obviously a drug of choice for the therapy of MDS since it has better therapeutic efficacies than Vidaza (azacitidine for injection) and Decitabine (in a class of medications called hypomethylation agents), the two US approved drugs, both on cytological evaluation and hematological improvement evaluation [49, 50]. Better yet, CDA-2 is, totally devoid of serious side effects, whereas Vidaza and Decitabine are proven carcinogens and very toxic to DNA [51-54]. MDS is a disease attributable entirely to CSCs as above described [28]. Thus, wound healing metabolites are proven drugs to display clinical efficacy against CSCs.

### Abnormal MEs as the Bullseye of Targeted Cancer Therapies

Cancer arises as a consequence of wound not healing properly due to the collapse of the functionality of Chemo-sur. Wound healing metabolites constitute the basis of Chemo-sur. Therefore, wound healing metabolites are most appropriate for cancer therapy [6]. Abnormal MEs are the target of wound healing metabolites, which should be also regarded as the target of cancer therapy. Selective cancer targets must be those specific to cancer. Abnormal MEs are also detectable in normal ESCs and PSCs. But the silencing of TET-1 makes abnormal MEs a unique abnormality of cancer. Therefore, abnormal MEs are a selective cancer target, and they are a selective target to all cancers [36].

An effective drug developed for a cancer can be applicable for the therapy of many other cancers, although some modifications may be necessary to take care of specific features of each cancer. Oncogenes and oncogene products constitute specific career targets too. Cancer therapies based on targeted therapies are always better therapies, because targeted therapies can avoid adverse side effects. In the past we set up disappearance of tumor as a criterion to favor selection of non-targeted destruction agents for cancer therapy. That practice must be dropped, because non-targeted destruction agents cannot solve cancer problem. They are the choice of cancer establishments to combat cancer in the past, but fail to win the battle. These agents cannot win the battle, because they are contra-indication. They create more wounds to aggravate the already bad situations. Their inability to take out CSCs, and the contribution to further damage the functionality of Chemo-sur laid the ground for inevitable recurrence and fatality. So even the fortunate few achieving complete remission are eventually succumbed to recurrence.

May be a very few early stage cancer patients whose functionality of Chemo-sur is not fatally damaged in the process can recover to subdue surviving CSCs. There is a clinical need to set up different criteria for the selection of cancer drugs. It is disclosed that induction of TD of CSCs and cancer cells provides a favorable criterion of selecting effective cancer drugs. TD of PSCs is the critical mechanism of perfect wound healing. TD of cancer cells is also a process that can negate the contribution of other cancer factors such as oncogenes, suppressor genes, and their products. Oncogenes and suppressor genes are cell cycle regulatory genes; they have important functions to play when cell is in cell cycle replicating. But if the cell is induced to undergo TD to exit cell cycle, they have no roles to play. So targeting abnormal MEs can have the effects to also targeting other cancer specific targets. Just like killing cancer cell also put to death all other cancer factors. Here it is disclosed a pharmaceutical composition and/or a method of targeting abnormal MEs to destabilize abnormal MEs for the perfection of wound healing to avoid ugly scar and cancer evolution.

### SUMMARY OF THE INVENTION

The present invention relates to destabilization of abnormal MEs to avoid ugly scar and cancer, and to combat cancer. We have discovered abnormal MEs as a pivotal cause of cancer. The abnormality of MEs is due to the association of MEs with telomerase. We were the first to propose Chemo-sur as a natural defense mechanism against cancer. In one embodiment, it is proposed that Chemo-sur is a natural mechanism to ensure perfect wound healing to avoid cancer. To ensure wound healing is the primary objective of Chemo-sur, and the avoidance of cancer is the natural consequence. Wound healing requires the proliferation of PSCs and the TD of PSCs. That makes the wound healing and the evolution of cancer so closely related. It takes only one single hit to silence ten-eleven translocation dioxygenase-1 enzyme (TET-1 enzyme) to convert PSCs to cancer stem cells (CSCs), which is well within the reach of PSCs equipped with abnormal MEs like cancer cells. TET-1 enzyme is responsible for the lineage transition of PSCs, which is eliminated in CSCs to block all differentiation programs. So CSCs and their progenies can only keep on perpetual proliferation that is the hallmark of cancer.

Therefore, the mechanism of wound healing is very importantly related to the evolution of cancer and the mitigation of cancer. Wounds are always healed perfectly without having to put up any effort, because we have the protection of Chemo-sur. If the functionality of Chemo-sur is destroyed due to pathological causes such as immunological or toxic-chemical disorders, then the proliferation of PSCs beyond the need to heal the wound can evolve into CSCs to make ugly scars, and even worse to progress to faster growing cancer cells by activation of oncogenes or inactivation of suppressor genes. PSCs and CSCs are very much alike. The only difference is that the TET-1 enzyme functions in PSCs, but is silenced in CSCs. They are both protected by drug resistance mechanism, so cytotoxic drugs and radiation cannot harm them. One of their major missions is to heal the wound. Wound healing metabolites are their natural partners, so wound healing metabolites can freely get into these cells to direct TD of PSCs for the completion of wound healing, and to put CSCs away. CDA-2 is a preparation of wound healing metabolites purified from urine, which is the drug of choice for the therapy of MDS, a disease attributable entirely to CSCs. Apparently wound healing metabolites are the best to heal wound perfectly, and to avoid cancer, and also to combat cancer. So we propose to make CDA formulations for the perfection of wound healing, and for cancer therapy, particularly against those cancers untreatable by cytotoxic drugs and radiation.

In one exempt embodiment of the present invention, CDA-WH ointment preparation is a 50% of AA: pregnenolone 1:1 mixture in the ointment preparation for perfect wound healing. In another embodiment, CDA-CSC is a formulation consisting of 24 µM of AA-32.3 µM of BIBR1532-14.3 µM pregnenolonge-1.24 µM of curcumin. In still another embodiment, CDA-MDS is a formulation consisting of 46.8 µM of AA-21.5 µM of pregnenolone. CDA-BT is a formulation consisting of 46.8 µM of AA-14.3 µM of pregnenolone-0.05 µM of pyrvinium pamoate, Further in an embodiment, CDA-Mel is a formulation consisting of 24 µM of AA-32.3 µM of BIBR1532-4.6 µM resveratrol. 10 g phenylacetylglutamine per day by oral administration of a capsule preparation is recommended to restore the functionality of Chemo-sur as a separate or additional treatment modality.

Wound healing and the evolution of cancer are closely related to involve PSCs as the critical common elements. It is hereby disclosed that Chem-sur is a natural defense mechanism against cancer and is a natural mechanism for the perfection of wound healing to avoid cancer. Wound triggers biological response that is good for wound healing, and immunological response that is bad for wound healing. The biological response involve the release of AA from membrane bound phosphatidyl inositol for the synthesis of PGs, which are essential to cause inflammation at the site of wound for the extravasation of Chemo-sur metabolites from inside of PSCs in order for PSCs to buildup to work on the repair. The functionality of Chemo-sur metabolites is responsible for the completion of the TD of PSCs to heal the wound. Thus, the functionality of Chemo-sur dictates the success of wound healing. Wounds are always healed perfectly without having to put up any effort in healthy people with Chemo-sur intact. The bad immunological response of wound can lead to the collapse of the functionality of Chemo-sur. In particular, TNF can cause cachexia symptom to result in excessive urinary excretion of Chemo-sur metabolites.

Without the safe guard of Chemo-sur to completely inducing the TD of PSCs, PSCs may evolve to become CSCs which may pile up to cause scar, or even worse to progress to faster growing cancer cells. Our disclosure of wound healing mechanism will enable us to design preparations of wound healing metabolites for the perfection of wound healing to avoid scar or ugly scar and the evolution of cancer. Based on the same tactic, we can also design preparations to take out CSCs and cancer cells to accomplish a permanent cure of cancer. CSCs are a subpopulation of cancer cells very resistant to toxic chemicals and radiation. Wound healing metabolites are the best candidates to take out this tough subpopulation. Evidently abnormal MEs are the target of wound healing metabolites for the perfection of wound healing, which are also one aspect of our invention. So we are in a unique position to formulate perfect cancer drugs that can take out both CSCs and cancer cells, and to restore the functionality of Chemo-sur for a permanent cure of cancer.

The present invention related to destabilization of abnormal MEs for the perfection of wound healing to avoid scar and cancer, and to combat cancer. Accordingly, some aspects of the present invention provide a composition of the chemicals or combination of chemicals as listed in Table 6 as Dls and in Table 7 as DHls for the preparation of CDA formulations with specific purposes such as prevention of scar, prevention of cancer, therapy of CSCs, therapy of MDS, therapy of malignant brain tumor, therapy of melanoma, and the like. In Table 6, PGJ2 is prostaglandin J2; 16,16-dimethylPGE2 is 16,16-dimethylprosglandin E2; PGE2 is prostaglandin E2; BicycloPGE2 is bicycloprostaglandin E2; and AA is arachidonic acid.

Some aspects of the present invention provide a method for administration of an active ingredient to a subject to destabilize abnormal methylation enzymes for the perfection of wound healing, comprising: administering to the subject an effective amount of a pharmaceutical composition comprising: a differentiation inducer, a differentiation helper inducer, or an anti-cachexia chemical as the active ingredient. In one embodiment, the subject is human. In another embodiment, the pharmaceutical composition has a form suitable to be administered orally, parenterally, or topically to the subject.

Some aspects of the present invention provide a method for administration of an active ingredient to a subject to destabilize abnormal methylation enzymes for taking out CSCs and/or cancer cells to accomplish a permanent cure of cancer, comprising: administering to the subject an effective amount of a pharmaceutical composition comprising: a differentiation inducer, a differentiation helper inducer, or an anti-cachexia chemical as the active ingredient.

Some aspects of the present invention provide a method for administration of an active ingredient to a subject to destabilize abnormal methylation enzymes for the perfection of wound healing, comprising administering to the subject an effective amount of a pharmaceutical composition comprising a differentiation inducer selected from a group consisting of prostaglandin J2, 16, 16-dimethylprostaglandin E2, prostaglandin E2, bicycloprostaglandin E2, arachidonic acid, BIBR1532, and boldine.

Some aspects of the present invention provide a method for administration of an active ingredient to a subject to destabilize abnormal methylation enzymes for the perfection of wound healing, comprising administering to the subject an effective amount of a pharmaceutical composition comprising a differentiation helper inducer of SAHH inhibitors selected from a group consisting of pyvinium pamoate, vitamin D₃, dexamethasone, testosterone, gugusterone, β-sitosterol, dehydroepiandrosterone, dihydrotestosterone, prednisolone, estradiol, progesterone, hydrocortisone, pregnenolone, and pregnenolone sulfate.

Some aspects of the present invention provide a method for administration of an active ingredient to a subject to destabilize abnormal methylation enzymes for the perfection of wound healing, comprising administering to the subject an effective amount of a pharmaceutical composition comprising a differentiation helper inducer of MT inhibitors selected from a group consisting of ethidium bromide, uroerythrin, hycanthone, and riboflavin.

Some aspects of the present invention provide a method for administration of an active ingredient to a subject to destabilize abnormal methylation enzymes for the perfection of wound healing, comprising administering to the subject an effective amount of a pharmaceutical composition comprising a differentiation helper inducer of MAT inhibitors selected from a group consisting of indolacetic acid, phenylacetylvaline, phenylacetylleucine, phenylacetylisoleucine, butyric acid, and phenylbutyric acid.

Some aspects of the present invention provide a method for administration of an active ingredient to a subject to destabilize abnormal methylation enzymes for the perfection of wound healing, comprising administering to the subject an effective amount of a pharmaceutical composition comprising a differentiation helper inducer of polyphenols selected from a group consisting of tannic acid, epigallocatechin gallate, resveratrol, curcumin, kuromanin, coumestrol, genisteine, pterostilbene, pyrogallol, silibinin, caffeic acid, ellagic acid, gallic acid, ferulic acid, and phloroglucinaol.

Some aspects of the present invention provide a method for administration of an active ingredient to a subject to destabilize abnormal methylation enzymes for the perfection of wound healing, comprising administering to the subject an effective amount of a pharmaceutical composition comprising a differentiation helper inducer of signal transduction and/or growth inhibitors selected from a group consisting of sutent, berberine, vorient, gleevec, metformin, As₂O₃, CoCl₂, and selenite.

Some aspects of the present invention provide a method for administration of an active ingredient to a subject to destabilize abnormal methylation enzymes for the perfection of wound healing, comprising administering to the subject an effective amount of a pharmaceutical composition comprising an anti-cachexia chemical, which comprises phenylacetylglutamine.

Some aspects of the present invention provide a method for administration of an active ingredient to a subject to destabilize abnormal methylation enzymes for the perfection of wound healing, wherein the perfection of wound healing comprises an indication of prevention of scar, modification of scar, treatment of cancer, or the like.

Some aspects of the present invention provide a method for administration of an active ingredient to a subject to destabilize abnormal methylation enzymes for the perfection of wound healing, wherein the perfection of wound healing comprises an indication of prevention of scar or treatment of cancer, the method comprising administering to the subject an effective amount of a pharmaceutical composition wherein the pharmaceutical composition comprises CDA-WH, which is an ointment preparation suitable for topical administration for the prevention of scar. In one embodiment, the preparation is 50% arachidonic acid:pregnenolone (1:1) in an ointment base.

Some aspects of the present invention provide a method for administration of an active ingredient to a subject to destabilize abnormal MEs for the perfection of wound healing, wherein the perfection of wound healing comprises an indication of prevention of scar or treatment of cancer, comprising administering to the subject an effective amount of a pharmaceutical composition, wherein the pharmaceutical composition comprises CDA-CSC, which is a parenteral preparation suitable for the treatment of cancer to direct terminal differentiation (TD) of cancer stem cells (CSCs) and cancer cells (CC), In one embodiment, the preparation is a mixture of 36 mg of arachidonic acid + 52 mg of BIBR1532 + 22.5 mg of pregnenolone + 2.3 mg of curcumin in 5 ml of saline solution as a single dose.

Some aspects of the present invention provide a method for administration of an active ingredient to a subject to destabilize abnormal methylation enzymes for the perfection of wound healing, wherein the perfection of wound healing comprises an indication of prevention of scar or treatment of cancer, comprising administering to the subject an effective amount of a pharmaceutical composition, wherein the pharmaceutical composition of CDA-CSC further comprises 10 g phenylacetylglutamine capsules.

Some aspects of the present invention provide a method for administration of an active ingredient to a subject to destabilize abnormal methylation enzymes for the perfection of wound healing, wherein the perfection of wound healing comprises an indication of treatment of MDS, comprising administering to the subject an effective amount of a pharmaceutical composition, wherein the pharmaceutical composition comprises CDA-MDS, which is a parenteral preparation suitable for the treatment of MDS. In one embodiment, the preparation is a mixture of 70.5 mg of arachidonic acid + 34 mg of pregnenolone in 5 ml of saline solution as a single dose. In other embodiment, the pharmaceutical composition of CDA-MDS further comprises 10 g phenylacetylglutamine capsules.

Some aspects of the present invention provide a method for administration of an active ingredient to a subject to destabilize abnormal methylation enzymes for the perfection of wound healing, wherein the perfection of wound healing comprises an indication of treatment of malignant brain tumor, comprising administering to the subject an effective amount of a pharmaceutical composition, wherein the pharmaceutical composition comprises CDA-BT, which is a parenteral preparation suitable for the treatment of malignant brain tumor. In one embodiment, the preparation is a mixture of 70.5 mg arachidonic acid + 22.5 mg of pregnenolone + 0.3 mg of pyrvinium pamoate in 5 ml of saline solution as a single dose. In other embodiment, the pharmaceutical composition of CDA-BT further comprises 10 g phenylacetylglutamine capsules.

Some aspects of the present invention provide a method for administration of an active ingredient to a subject to destabilize abnormal methylation enzymes for the perfection of wound healing, wherein the perfection of wound healing comprises an indication of treatment of malignant brain tumor, comprising administering to the subject an effective amount of a pharmaceutical composition, wherein the pharmaceutical composition comprises CDA-Mel, which is a parenteral preparation suitable for the treatment of melanoma. In one embodiment, the preparation is a mixture of 36 mg of arachidonic acid + 52 mg of BIBR1532 + 52.5 mg of resveratrol in 5 ml of saline solution as a single dose. In other embodiment, the pharmaceutical composition of CDA-Mel further comprises 10 g phenylacetylglutamine capsules.

### BRIEF DESCRIPTION OF THE DRAWINGS

Additional objects and features of the present invention will become more apparent and the disclosure itself will be best understood from the following Detailed Description of the Exemplary Embodiments, when read with references to the accompanying drawings.

FIG. 1 shows effect of testosterone administration on the sedimentation profiles of MEs from long-term hormone-depleted prostate.

FIG. 2 shows identification of the tumor factor of abnormal tumor MEs as the catalytic subunit of telomerase.

FIG. 3 shows the quantitative analysis of peptide preparations.

FIG. 4 shows chemo-prevention of aflatoxin B1 induced hepatocarcinogenesis by A10 (A10 also known as “phenylacetylglutamine”).

FIG. 5 shows the sensitivity of CSCs subpopulation toward cytotoxic drugs and CDA-2.

FIG. 6 shows induction of TD by CDA-2.

FIG. 7 shows therapeutic efficacy of CDA-2 on MDS.

FIG. 8 shows the relationship of wound to the development of cancer.

FIG. 9 shows the role of methylation enzymes in the regulation of cell replication, differentiation, and apoptosis.

### DETAILED DESCRIPTION OF THE EXEMPLARY EMBODIMENTS

The preferred embodiments of the present invention described below relate particularly to the preparation of pharmaceutical compositions to destabilize abnormal MEs for the perfection of wound healing to avoid ugly scar and cancer evolution, and more particularly, for the use to take out both cancer cells and cancer stem cells to accomplish a permanent cure of cancer thereof. While the description sets forth various embodiment specific details, it should be understood that the description is illustrative only and should not be construed in any way as limiting the invention. Furthermore, various applications of the invention, and modifications thereto, which may occur to those who are skilled in the art, are also encompassed by the general concepts described below.

### Role of MEs in the Regulation of Cell Replication, Differentiation, and Apoptosis

MEs play a critical role on the regulation of cell replication, differentiation, and apoptosis by virtue of the fact that DNA MEs control the expression of tissue specific genes [56], and pre-rRNA MEs control the production of ribosome [57], which in turn dictates the commitment of cells to initiate replication [58]. If enhanced production of ribosome is locked in place, it becomes a factor to drive carcinogenesis [59]. Biological methylation is mediated by a ternary enzyme complex consisting of MAT-MT-SAHH [5]. These enzymes must be in a ternary enzyme complex to become stable and functional. In the monomeric state, individual enzymes are quickly inactivated. SAHH is the most unstable enzyme, followed by MT, and then MAT. MTs in the monomeric state have a great tendency to be converted into nucleases to trigger apoptosis. SAHH requires a stabilizing factor such as a steroid hormone to assume confirmation favorable for the formation of dimeric complex with MT.

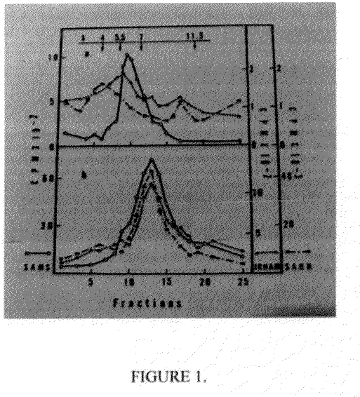

The MT-SAHH dimmer has a molecular size similar to MAT suitable for the formation of a ternary enzyme complex. In normal cells, the stabilizing factors of SAHH, either steroid hormones or related factors as shown in the FIG. 1 of the drawing, are the dominant factors to modulate MEs. in cancer cells and telomerase expressing cells such as ESCs and PSCs, MEs are associated with telomerase to turn MEs exceptionally stable and active to block TD. The abnormal MAT-SAHH isozyme pair display Kₘ values 7-fold higher than the normal isozyme pair [5, 60]. The higher Kₘ value is the reason for abnormal MEs to display exceptional stability and activity. According to Prudova et al., S-adenosylmethionine (AdoMet) could protect protein against protease digestion [61]. It has been shown by Chiba et al. that the pool sizes of AdoMet and S-adenosylhomocysteine (AdoHcy) shrunk greatly when HL-60 cells were induced to undergo TD. These observations strongly support our findings that abnormal MEs are an important factor of cancer. Evidently abnormal MEs also play an important role to buildup cell mass for the development of fetus and for wound healing.

### Abnormal MEs of Cancer Cells

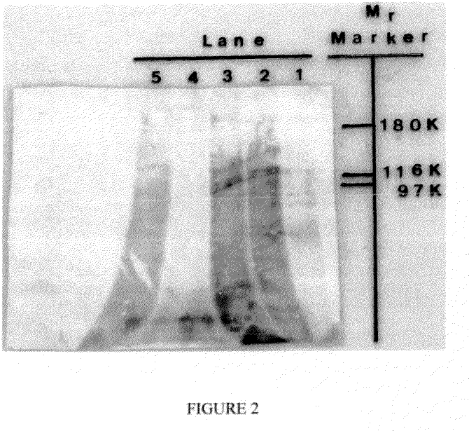

Abnormal MEs of cancer cells are due to the association of a tumor factor with MEs that change the kinetic properties and the regulation of MEs. We have identified the tumor factor as the catalytic subunit of telomerase as shown in FIG. 2 of the drawing. The tumor factor can be dissociated from MAT^{LT} by exposing MAT^{LT} to a buffer made up by 0.05 M Tris, PH 8.2, 5 mM EDTA, 5 mM mercaptoethanol. Upon rechromatography of dissociated enzyme it yielded an enzyme preparation without reacting bands with telomerase antibody, which displayed a Kₘ value of 3 µM methionine similar to that of the liver MAT^{L}. Apparently abnormal MEs are common to all human cancers, as all xenografted human cancer tissues displayed abnormal MEs in the pH 5 precipitates of cytosol extracts as shown in Table 1. Even a surgical specimen had the abnormal MEs. Clearly the abnormality of MEs is not an artifact of xenograft. The activity of MAT^{LT} appears to correlate very well with the growth rate. Abnormal MEs were also detectable in experimental animal tumors, starting from preneoplastic hyperplastic nodules [35]. This was an indication that CSCs were originated from PSCs, and then progressed to faster growing cancer.

### Chemo-Sur as a Natural Mechanism for the Perfection of Wound Healing to Avoid Cancer

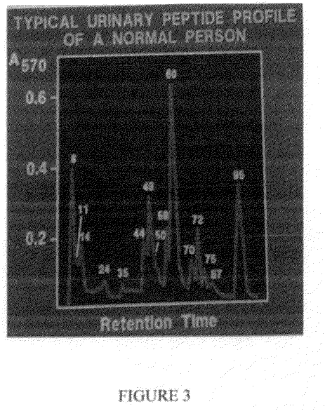

The use of urinary peptides for cancer therapy was initiated by Burzynski et al. who discovered that urinary peptides had selective inhibitory activity against cancer cells [63, 64]. Peptide analysis was carried out to find out the answer why cancer evolves in the presence of peptides effective to control the growth of cancer cells. Quantitative measurement of plasma and urinary peptides by a procedure shown in FIG. 3 of the drawing indicated that cancer patients showed deficiency of plasma peptides, and excessive urinary excretion as plasma/urinary peptide ratios of cancer patients were mostly below that of healthy persons as shown in Table 2 and Table 3.

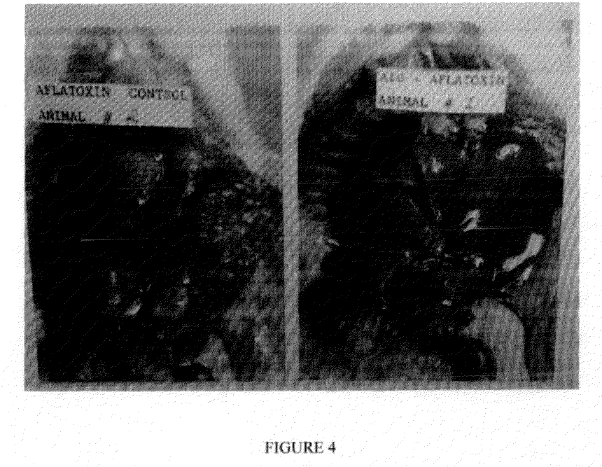

Peptides are actually the surrogate molecules of the active components of urinary metabolites displaying antitumor effects. We have found metabolites which were not peptides to display impressive activity as Dls [4, 65]. We have also found that phenylacetylglutamine was an effective chemo-preventive agent which did not have antitumor effect. Phenyacetylglutamine is a major urinary metabolite. It is a major chemical component of Antineoplaston preparations and CDA-2, the urinary preparations used for cancer therapy. Phenylacetylglutamine was effective to prevent excessive urinary excretion of low molecular weight metabolites [1]. In essence, phenylacetylglutamine is an effective anti-cachexia chemical to prevent the loss of metabolites with antitumor activity. By preventing the loss of antitumor chemicals, it could have impressive chemo-prevention effect on aflatoxin B1 induced hepatocarcinogenesis as shown in FIG. 4 of the drawing. It is quite remarkable that an innocent chemical such as phenylacetylglutamine could display such a strong effect to prevent aflatoxin B1, which is a very potent hepatocarcinogen, induced hepatocarcinomas. The protection of the functionality of Chemo-sur is really important to stay free of cancer [1, 13, 37]. The use of phenylacetylglutamine to restore the functionality of Chemo-sur is really a small price to gain a giant benefit.

### Metabolites Involved in Chemo-Sur to Ensure Wound Healing Are Dls and DHIs

Acidic peptides, OA-0.79, and PP-0 were three major Dis found in Antineoplaston A5, which were urinary metabolites retained by C18. XAD-16 could not retain peptides; therefore OA-0.79 and PP-0 were two major Dls of CDA-2. As shown in Table 4, Dls of urinary metabolites could selectively eliminate telomerase from abnormal MTA^{LT} to turn this abnormal isozyme to become normal isozyme MAT^{L}, whereas on MAT^{L} they did not have any appreciable effect. Thus, wound healing metabolites act selectively to induce TD of cells with abnormal MEs. OA-79 is very likely a mixture of AA and pregnenolone, and PP-0 is membrane fragments containing AA which could be released by hydrolysis in 0.5 M NaOH at 37° C. [11, 65]. We do not know the identity of acidic peptides. Attempts were made to synthesize acidic pentapeptides picked from hemoglobin to test their activity as Dls. None showed impressive activity [65].

DHI was our invention, which was the term we created [66]. DHls are inhibitors of individual enzymes of MEs. They provide helping role to boost the activity of Dls as shown in the Table 5. Rl_{0.5} was the method we developed for the measurement of potency as DHI [67]. Inhibitors of MT and SAHH are better than inhibitors of MAT to function as DHIs [68, 69].

### CDA-2 as a Perfect Cancer Drug

Perpetual cell replication is the hallmark of cancer. There are multiple issues involved to make cancer cells to replicate perpetually. The production of inflammatory cytokines caused by wound leads to leaking renal tubules to result in the collapse of Chemo-sur. The collapse of Chemo-sur enables PSCs to keep on replicating to evolve into CSCs by silencing TET-1 enzyme that totally eliminate differentiation capability of CSCs. The progression to activate oncogenes or to inactivate suppressor genes then promotes CSCs to become faster growing cancer cells.

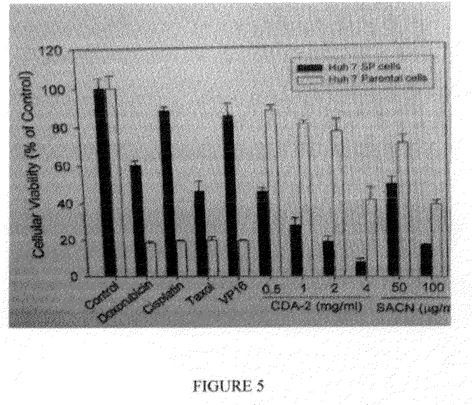

A perfect cancer drug must be able to resolve all issues contributing to the evolution of PSCs to become faster growing cancer cells. CDA-2 is such a perfect cancer drug. CDA-2 was the invention of Liau [48], which was a preparation of urinary metabolites retained by XAD-16. It contains AA as the major DI, pregnenolone, uroerythrin, and steroid metabolites as DHIs, and phenylacetylglutamine as an anti-cachexia chemical. The active ingredients of CDA-2 are wound healing metabolites tolerable to PSCs and CSCs. It has shown remarkable efficacy on CSCs as shown in FIG. 5 of the drawing. The results showed that parental cells were sensitively inhibited by cytotoxic drugs, whereas CSCs subpopulation was resistant to cytotoxic drugs. On the contrary, CSCs subpopulation was preferentially inhibited by CDA-2, whereas the viability of parental cells was not affected during the 48 h of incubation with CDA-2.

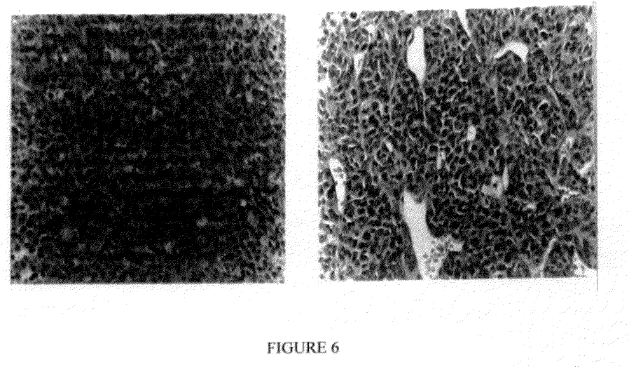

Therapeutic endpoint of CDA-2 is TD. Induction of TD takes two cell cycles to complete. The tumor may increase in size slightly and then stop to assume differentiated structure as shown in the FIG. 6 of the drawing. CDA-2 can really turn cancer cells into functional cells. MDS is a disease attributable entirely to CSCs [28]. The therapy requires differentiation of pathological CSCs to become functional erythrocytes that is the measure of hematological improvement. CDA-2 is obviously better than Vidaza and Decitabine in this regard. It does not have toxic effect on DNA to cause carcinogenesis [51] and DNA damages [52-54]. CDA-2 is the drug of choice for the therapy of MDS. CDA-2 was approved for the therapy of breast cancer, non-small cell lung cancer and primary hepatoma by the Chinese FDA in 2004 and for the therapy of MDS in 2017. We are now relying on CDA-2 as the prototype to develop synthetic CDA formulations for a permanent cure of cancer.

### Abnormal MEs as the Bullseye of Targeted Cancer Therapies

Targeted therapy is always a better therapy than non-targeted therapy, because it can avoid adverse effects. But non-targeted therapy dominates cancer therapy in the past, because cancer establishments set up a rule to put the disappearance of tumor as a criterion for the evaluation of efficacy of cancer drug. This is the most damaging mistake committed by the cancer establishments to favor destructive agents to kill cancer cells for cancer therapy. MDS is a clear example that cancer arises as a consequence of wound not healing properly. The functionality of Chemo-sur must be badly damaged for cancer to appear. The use of toxic agents to create more wounds to further damage the functionality of Chemo-sur is contra-indication. If the killing of cancer cells can include CSCs, it may be an acceptable therapy. The functionality of Chemo-sur may gradually recover to subdue PSCs from evolving into CSCs to cause another cancer problem. The inability of cytotoxic agents to take out CSCs and the contribution to damage Chemo-sur laid the ground for inevitable recurrence and fatality. This is the reason why destructive therapies are so effective to kill cancer cells, but not many cancer patients are saved. Cancer mortalities remain at old time high worldwide.

Targeted therapy is a better choice of cancer therapy. Abnormal MEs may not be considered as a specific cancer target, since abnormal MEs also happen in normal stem cells such as ESCs and PSCs. The silence of TET-1 enzymes qualifies abnormal enzymes as a cancer target [14-18]. Actually, abnormal MEs are the best cancer target, because when this target is solved, it can also put to rest all other cancer targets. The other cancer targets include gene abnormalities and their products. Oncogenes and suppressor genes are cell cycle regulatory genes. They have important functions to play when cell is in cell cycle replicating. But if the cell exits cell cycle to undergo TD, they have no role to play. So a stroke to solve abnormal MEs can also put to rest all other important issues contributing to the perpetual proliferation of cancer cells. It is the ultimate solution of cancer.

There is a big problem of this targeted therapy. The tumor will not go away. We have to set up different criteria for the evaluation of therapeutic efficacy. Disappearance of circulating CSCs is a valid criterion. Restoration of the functionality of Chemo-sur by quantitative assay of plasma and urinary wound healing metabolites can provide clue of effectiveness.

### Synthetic CDA-Formulations as Perfect Drugs for Wound Healing and Cancer Therapy

Metabolites active as Dls and DHIs are the creation of the nature to ensure wound healing to avoid cancer and to beat cancer. The best strategy of cancer therapy is to follow the natural course that heals the wound. The natural course to heal the wound is to enable the proliferation of PSCs and to direct the TD of PSCs to complete the wound healing. TD of replicating PSCs becomes a critical issue of wound healing. If the functionality of Chemo-sur is good, wound can be healed perfectly. If not good, PSCs can evolve into CSCs to keep on replicating that make ugly scar if CSCs are eventually directed to undergo TD. If there is not enough wound healing metabolites to direct the TD of PSCs, PSCs may evolve to become CSCs, which can make ugly scar, and worse can progress to faster growing cancer cells by activation of oncogenes or inactivation of suppressor genes. Inactivation of suppressor genes is easier to accomplish by CSCs, simply by silencing suppressor genes with their superbly active MEs. So the elimination of abnormal MEs is really the key to the success of cancer therapy.

If abnormal MEs are not eliminated early on, they can create immense damages. Damages such as aberrant methylations to knock out expression of suppressor genes and repair genes, which are essential factors for cancer progression. Cytotoxic drugs by slowing down DNA synthesis encourage aberrant DNA methylation [70, 71]. Aberrant DNA methylation is a reverse process of lineage transition carried out by TET-1 enzyme, thus pushing cancer cells to revert to more primitive lineages such as CSCs to resist cancer therapy. Antineoplaston components, and also CDA-2 components, could prevent aberrant DNA methylation to halt cancer progression [72]. Therefore, targeting abnormal MEs is a correct strategy for cancer therapy. It is the choice of the nature.

In the second example and exempt embodiment, the present invention provides a method of treatment to prevent ugly scar due to insufficient wound healing metabolites naturally produced. We propose to use AA + pregnenolone in the proportion of ED₂₅ of AA: 3xRl_{0.5} of pregnenolone as the basic composition. That comes to a mixture of 24 µM AA and 21.5 µM pregnenolone. The Mᵣ of AA is 304.5 and the Mᵣ of pregnenolone is 316.5. It is about equal weight of AA and pregnenolone. Use equal weight of AA and pregnenolone to combine with an equal weight of ointment base to make 50% of AA: pregnenolone (1:1) preparation of wound healing ointment. We designate the preparation as CDA-WH ointment.

In the third example and exempt embodiment, the present invention provides a method of treatment to take out CSCs. We propose to use ED₂₅ of AA + ED₂₅ of BIBR1532 + 2xRl_{0.5} of pregnenolone + Rl_{0.5} of curcumin as the CDA-CSC formulation. This is a mixture of 24 µM AA + 32.3 µM BIBR1532 + 14.3 µM pregnenolone + 1.24 µM of curcumin. On the weight basis the mixture is 7.2 mg of AA + 10.4 mg of BIBR1532 + 4.5 mg of pregnenolone + 0.46 mg of curcumin to make one dosage/L of blood that can yield 100% induction of TD of cancer cells, CSCs included. The average blood volume is 5 liters per person. Therefore, we need to multiply the amounts by a factor of 5 to produce one dose. Dissolve 36 mg of AA + 52 mg of BIBR1532 + 22.5 mg of pregnenolone + 2.3 mg of curcumin in 5 ml of saline solution to make one dose/day. Since water solubility of pregnenolone and curcumin is not good. Liposomal technique may be considered to increase their solubility and bioavailability. For the treatment of human cancer, we recommend the use of 10 g/day of phenylacetylglutamine in capsules per oral to restore the functionality of Chemo-sur as a separate treatment modality. The treatment can be discontinued when plasma/urine ratios of wound healing metabolites such as peptides, uroerythrin, pregnenolone, or AA have reached the level of normal persons. CDA-CSC can also be used on surgical patients to help healing the wound perfectly, particularly on cancer patients undergoing surgery. Chemo-sur of cancer patients has been damaged badly for cancer to show up. Preparation such as CDA-CSC is very helpful to heal wound and to prevent metastasis.

In the 4^{th} example and exempt embodiment, the present invention provides a method of treatment on MDS. CDA-2 is obviously a drug of choice for the therapy of MDS, which contain AA as the major DI and pregnenolone as the major DHI. We propose to use ED₅₀ of AA + 3xRl_{0.5} of pregnenolone as the CDA-MDS formulation. This is a mixture of 46.8 µM of AA + 21.5 µM of pregnenolone. On the weight basis the mixture is 14.1 mg of AA + 6.8 mg of pregnenolone. Multiply these amounts by a factor of 5 to produce a single dose. Dissolve 70.5 mg of AA and 34 mg of pregnenolone in 5 ml of saline as above described to make a single dose. The use of 10 g/day of phenylacetylglutamine is also recommended.

In the 5^{th} example and exempt embodiment, the present invention provides a method of treatment on malignant brain tumors. Malignant brain tumors are notoriously resistant to cytotoxic chemotherapy and radiation therapy, because they are enriched in CSCs [75]. Malignant brain tumors responded very well to phenylbutyrate, a modest DHI we discovered [66], which was dexterous employed by Burzynski to successfully cure untreatable malignant tumors [76-79]. Brain compartment is full of lipid materials, some of which may have similar function as AA. Therefore, even a modest DHI such as phenylbutyrate can have unbelievable therapeutic effect. We propose to use ED₅₀ of AA + 2xRl_{0.5} of pregnenolone + 4xRl_{0.5} of pyrvinium pamoate as the CDA-BT formulation. This is a mixture of 46.8 µM of AA +14.3 µM of pregnenolone + 0.05 µM pyrvinium pamoate. On the weight basis the mixture is 14.1 mg of AA + 4.5 mg of pregnenolone + 0.06 mg pyrvinium pamoate. Multiply these amounts by a factor of 5 to produce a single dose. Dissolve 70.5 mg of AA + 22.5 mg of pregnenolone + 0.3 mg of pyrvinium pamoate in 5 ml of saline solution as above described to make a single dose. The use of 10 g/day of phenylacetylglutamine is also recommended.

In the 6^{th} example and exempt embodiment, the present invention provides a method of treatment on melanoma. Melanoma is a tough untreatable cancer like malignant brain tumor because of enrichment of CSCs. Melanoma expresses a high level of hypoxia inducible factor which is a transcription factor of telomerase [80]. Resveratrol is a good inhibitor of HIF. We propose to use ED₂₅ of AA + ED₂₅ of BIBR1532 + 4xRl_{0.5} of resveratrol as CDA-Mel formulation. This is a mixture of 24 µM of AA + 32.3 µM of BIBR1532 + 4.6 µM resveratrol. On the weight basis the mixture is 7.2 mg of AA + 10.4 mg of BIBR1532 + 10.5 mg of resveratrol. Multiply these amounts by a factor of 5 to produce a single dose. Dissolve 36 mg of AA + 52 mg of BIBR1532 + 52.5 mg of resveratrol in 5 ml of saline solution as above described to make a single dose. The use of 10 g/day of phenylacetylglutamine is also recommended.

In summary, we have invented abnormal MEs as a pivotal cause of cancer. The abnormality is due to the association of telomerase with MEs. We have also invented Chemo-sur as a natural mechanism to ensure wound healing to avoid cancer. If the functionality of Chemo-sur is destroyed by chronic wounds, the proliferation of PSCs is likely to keep on to evolve into CSCs and then to progress to faster growing cancer cells by the activation of oncogenes or the inactivation of suppressor genes. There are multiple issues involved to cause cancer cells to proliferate perpetually. The solution of cancer is not as simple as to kill sensitive cancer cells. We have tried that to use very toxic chemicals and radiation. They wiped out sensitive cancer cells all right, but they cannot take out CSCs. Their contribution to further destroy the functionality of Chemo-sur laid the ground for inevitable recurrence and fatality. The perfect solution of cancer is to follow the course of the nature to heal wound.

The perfect course to heal the wound is to target abnormal MEs to direct TD of PSCs, which are closely related to CSCs. Abnormal MEs are not only critical to CSCs. They are also essential for the promotion of malignant growth. The same strategy to take out CSCs can also be effective to take out faster growing cancer cells. The therapeutic endpoint is TD of cancer cells that won’t make tumor to disappear. We have to set up different criteria for the evaluation of therapeutic efficacy based on the induction of TD of cancer cells. The solution of cancer is a battle between the wisdom of the nature and the wisdom of cancer establishments. Is the disappearance of tumor mass so important? The cancer establishments have failed to save the life of cancer patients in the past; they should give the way to the nature to save the life of cancer patients [73, 74].

FIG. 1 shows the effect of testosterone administration on the sedimentation profiles of MEs from long-term hormone-depleted prostate. Ten weeks after hypophysectomy, rats were divided into two groups. One group (b) received daily sc injection of I mg testosterone propionate dissolved in 0.25 ml of sesame oil for 3 days. The other group (a) was control without treatment. Cytosol extracts were prepared from prostates of each group with 5 volume of extraction buffer 0.05 M Tris HCl, pH 7.5, 50 mM KCl, 0.5 mM MgCl₂. 0.1 ml of cytosol extracts were layered onto 5-20% linear sucrose density gradient for sedimentation analyses. Enzymes of testosterone depleted cytosol were predominantly in the monomeric forms, with SAMS (namely MAT), tRNAM (namely MT), and SAHH sedimenting as 6.3 S, 5.5 S and 4 S, respectively. Enzymes of testosterone treated cytosol showed these enzymes sedimenting as a single peak of 8 S, the intact ternary MEs.

FIG. 2 shows identification of the tumor factor of abnormal tumor MEs as the catalytic subunit of telomerase. MAT enzyme preparations were purified by DEAE-agarose chromatography from pH 5 precipitates of cytosol of rat liver or rat Novikoff hepatoma, using KCI gradient of 0.05-0.5 M to elute enzyme. MAT usually showed up as a single peak at 0.35 M KCI eluant. Lane 1 is the electrophoresis profile of rat liver MAT^{L}, which does not have hTERT antibody reacting band. Lane 2 is that of rat Novikoff hepatoma MAT^{LT}, which shows double reacting bands at 120 K and 143 K. 120 K is hTERT, and 143 K is hTERT plus an associated protein of 23 K. The enzyme preparation of Lane 2 was divided into two portions, one dialyzed with 0.05 M Tris, pH 7.5, 5 mM MgCI₂, and purified again by DEAE-agarose chromatography using the buffer containing 5 mM MgCl₂. Lane 3 is the electrophoresis profile of this enzyme preparation, which again shows double reacting bands at 120 K and 143 K. Enzyme preparations with positive hTERT reacting bands always displayed Kₘ value of 22 µM methionine. The other half of Lane 2 was dialyzed with 0.05 M Tris, pH 8.2, 5 mM EDTA, 5mM mercaptoethanol, and repurified by DEAE-agarose chromatography using the dialysis buffer. There was an inactive protein band coming off around 0.1 M KCI. Lane 4 is the electrophoresis profile of this inactive protein, which does not have hTERT reacting band. Lane 5 is the active enzyme preparation which does not have hTERT antibody reacting band like that of liver enzyme. Enzyme without hTERT reacting band displayed Kₘ value of 3 µM methionine.

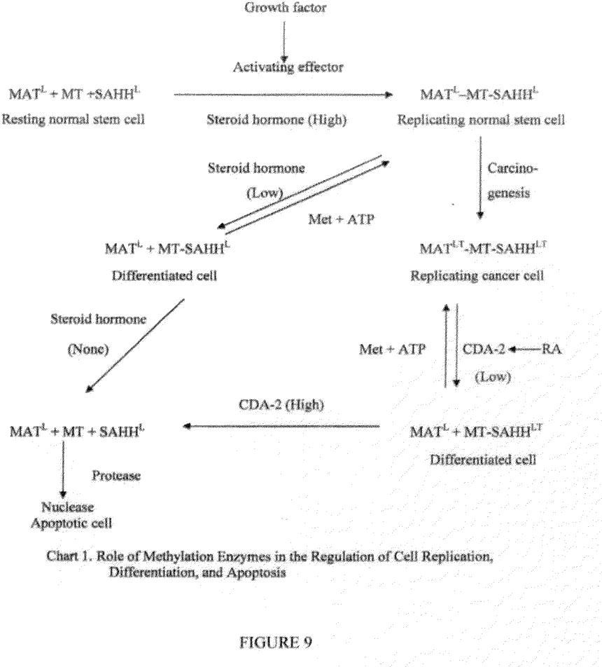

FIG. 9 shows the role of methylation enzymes in the regulation of cell replication, differentiation, and apoptosis. MEs are under the regulation of a stabilizing factor of SAHH which is the most unstable enzyme. The stabilizing factor is either steroid hormone in case of steroid hormone target tissues or similar factor produced under the direction of growth hormone. These stabilizing factors direct the formation of MT-SAHH dimer which has a similar molecular size as MAT. The MT-SAHH dimer is then in a position to form ternary MEs to become stable and functional entity of MEs. The dysfunction of MEs proceeds in the opposite direction. Dissociation of MT-SAHH dimer occurs at extreme depletion of the stabilizing factor. When MTs are in the monomeric state, they have a tendency to be converted into nucleases to trigger apoptosis to cause the involution of hormone-depleted tissues. MEs of cancer cells are abnormal due to association with telomerase to block TD. CDA-2 is a preparation of wound healing metabolites purified from urine that can eliminate telomerase from abnormal MEs. Retinoic acid (RA) can induce cells to produce oligoisoadenylate to function as CDA-2 to destabilize abnormal MEs.

The action of RA requires a RA receptor. Thus, only cancer cells expressing RA receptor can respond to RA therapy, whereas CDA-2 can affect all cancers. As shown in Table 1, the symbols are: SAMS is MAT. Low Kₘ is the MAT^{L} isozyme with a Kₘ value of 3 µM methionine. The intermediate Kₘ is the MAT^{LT} isozyme with a Kₘ value of 20 µM methionine.

Table 1

SAMS isoenzymes of human malignant tumors’

| Tissues | SAMS, V, nmol/min/8 tissue | | | | nude mice passage | | Tumor size. mm³ /any |
|---|---|---|---|---|---|---|---|
| | Low K. | | Intermediate K. | | | | |
| | Cellsol | pH-5 ppt | Cellsol | pH-5 ppt | No. | Days | |
| Melanoma, black | 1.11 | 0.48 | 4.03 | 1.43 | 1 | 120 | 45 |
| Melanoma, black | 0.67 | 0.25 | 4.35 | 1.93 | 10 | 38 | 171 |
| Melanoma, light brown | 0.61 | 0.27 | 8.76 | 1.49 | 3 | 60 | 84 |
| Melanoma, light brown | 0.97 | 0.33 | 4.10 | 2.37 | 18 | 80 | 183 |
| Melanoma, while | 0 | 0 | 7.60 | 3.02 | 3 | 29 | 822 |
| Melanoma, white | 0 | 0 | 8.44 | 3.74 | 15 | 29 | 335 |
| Colon carcinoma | 1.81 | 0.62 | 4.86 | 2.61 | 7 | 64 | 144 |
| Colon carcinoma HT-29 | 1.47 | 0.55 | 5.95 | 2.75 | 19 | 42 | 176 |
| Colon carcinoma | 0 | 0 | 5.56 | 2.84 | 11 | 28 | 318 |
| Colon carcinoma | 0 | 0 | 8.66 | 3.48 | 15 | 36 | 485 |
| Lung carcinoma | 0 | 0 | 8.75 | 8.70 | 84 | 16 | 254 |
| Lung carcinoma | 0 | 0 | 8.80 | 8.85 | 25 | 22 | 379 |
| Mammary carcinoma HBL-100 | 1.23 | 0.48 | 2.72 | 1.02 | 1 | 112 | 27 |
| Mammary carcinoma | 1.74 | 0.67 | 4.55 | 2.12 | 41 | 28 | 100 |
| Mammary carcinoma | 1.25 | 0.50 | 5.22 | 2.22 | 4 | 76 | 109 |
| Cervical carcinoma | 1.89 | 0.49 | 2.90 | 0.91 | 11 | 57 | 49 |
| Cervical carcinoma | 1.24 | 0.42 | 3.28 | 1.89 | 15 | 52 | 98 |
| Cervical carcinoma | 0 | 0 | 4.45 | 1.87 | 10 | 50 | 145 |
| Ovary carcinoma | 1.82 | 0.56 | 2.52 | 1.02 | 1 | 169 | 19 |
| Ovary carcinoma | 1.12 | 0.42 | 8.37 | 1.57 | 3 | 79 | 54 |
| Ovary carcinoma | 1.74 | 0.65 | 5.56 | 2.05 | 3 | 93 | 59 |
| Hepatoma | 1.78 | 0.62 | 8.27 | 1.17 | 6 | 128 | 58 |
| Hepatoma | 1.97 | 1.18 | 4.65 | 1.89 | 1 | 37 | 92 |
| Hepatoma | 1.83 | 0.45 | 6.78 | 2.50 | 9 | 66 | 280 |
| Sarcoma | 0.92 | 0.34 | 1.87 | 0.71 | 8 | 110 | 95 |
| Sarcoma | 0 | 0 | 5.20 | 2.08 | 7 | 64 | 385 |
| Stomach carcinoma | 0 | 0 | 7.44 | 8.40 | 8 | 50 | 162 |
| Lymphoma U-1285 | 0 | 0 | 6.95 | 2.98 | 11 | 49 | 166 |
| Nasopharyngeal carcinoma | 1.16 | 0.24 | 1.68 | 0.72 | 17 | 64 | 82 |
| Melanoma; white, surgical specimen | 2.42 | 0.78 | 3.98 | 1.72 | | | |

• Each tumor was initiated from the tumors of individual patients, except colon tumor HT-29, mammary carcinoma HBL-100, and lymphoma U-1285, which were initiated from established call lines. The passage in nude mice. duration, and tumor size per day were as indicated, SAMS from cell-soluble fraction (Cellsol) and pH-6 precipitate (pH-6 ppt) were partially purified by DEAE-cellulose chromatography as previously described (8, 3). Kₘ and Vₘₐₓ of SAMS were obtained from Lineweaver-Burk plots as previously described (2, 8). Dash=no data available (not passed through nude mice).

FIG. 3 shows the quantitative analysis of peptide preparations. Plasma was initially deproteinzed with 0.25 volume of 10 mg/ml sulfosalicylic acid, and the precipitate was removed by centrifugation. Deproteinzed plasma and urine were separated purified through C18 column. Peptides were eluted with 80% methanol, which was lyophilized and redissolved in water for peptide analysis. Peptide analysis was carried out by HPLC on a column of sulfonated polystyrene developed by Glenco Scientific Inc. of Houston, TX. The ninhydrin reacted color product was passed through a double-beam spectrophotometer for the detection of absorbance at 570 nm and 440 nm. The absorption profiles were recorded automatically on a chart and by an integrator. The integrated data were converted into equivalent amounts of amino group using standard amino acids as the reference.

Table 2

Plasma peptide levels of cancer patients

| Plasma levels (nmoles/ml) | No. of patients | % Distribution |
|---|---|---|
| 60-75 | 10 | 9.2 |
| (normal) | | |
| 50-60 | 15 | 13.9 |
| 40-50 | 22 | 20.4 |
| 30-40 | 30 | 27.8 |
| 20-30 | 27 | 25.0 |
| 10-20 | 4 | 3.7 |

Table 3

Plasma/urine peptide ratios of cancer patients

| Plasma/urine Ratios | No. of patients | % Distribution |
|---|---|---|
| 0.8-0.83 | 2 | 1.8 |
| (normal) | | |
| 0.6-0.8 | 7 | 6.5 |
| 0.4-0.5 | 18 | 16.7 |
| 0.2-0.4 | 38 | 35.2 |
| 0.1-0.2 | 24 | 22.2 |
| 0.02-0.1 | 19 | 17.6 |

(Note: Peptide analysis was conducted as described in FIG. 3. The plasma peptide concentration was nmole/ml and that of urinary peptide concentration was nmole/mg creatine.)

FIG. 4 shows chemo-prevention of aflatoxin B1 induced hepatocarcinogenesis by A10. A10, namely phenylacetylglutamine in this invention, was given to the group of rats feeding the diet containing 1% A10. The control group of rats was fed the diet without A10. Aflatoxin B1 dosing was initiated 8 days after A10 feeding at the dose of 25 µg in 50 µl DMSO/rat/day, 5 days weekly for 8 consecutive weeks. Multiple lesions of varying sizes and shapes of hepatocarcinomas were visible on the surface of control liver; whereas A10 treated liver did not show carcinoma lesions.

Table 4

Selective inhibition of MAT^{LT} by Dls

| Dls, mg/ml | | MAT^{LT} | | MAT^{L} |
|---|---|---|---|---|
| | Km | Vmax (^{%}) | Km | Vmax (%) |
| None | 20.3 | 11.1 (100) | 3.1 | 0.89 (100) |
| Acidic peptides, 0.1 | 3.7 | 5.8 (52) | 3.1 | 0.89 (100) |
| 0.2 | 3.3 | 5.5 (49) | 3.1 | 0.82 (93) |
| OA-0.79, 0.2 | 3.3 | 7.3 (66) | 3.1 | 0.85 (96) |
| 0.4 | 3.0 | 5.0 (45) | 3.0 | 0.78 (88) |
| PP-0, 0.5 | 3.2 | 7.0 (63) | 3.1 | 0.78 (88) |
| 1.0 | 3.1 | 4.7 (42) | 3.1 | 0.76 (85) |

Dls was purified from antineoplaston A5, which was a preparation of urinary metabolites retained by C18 and eluted with 80% methanol. Methanol was removed by rotary evaporator. The residue was dissolved in water, pH 7 to make antineoplaston A5. When pH of antineoplaston was adjusted to 2, acidic peptides remained in the supernatant. Acidic peptides were retained by Doxex I resin, and eluted with NaCl gradient between 0.75-1.5 M NaCl. NaCl was removed by C18. OA-0.79 and PP-0 were purified from the pH 2 precipitate by gel filtration on AcA202 column to collect Kₐᵥ=0 as PP-0, and Kₐᵥ=0.79 as OA-0.79. MAT^{LT} and MAT^{L} were enzyme preparation purified from pH 5 precipitate of cytosol extract of Novikoff hepatoma and rat liver, respectively, by DEAE agarose chromatography. Kₘ and Vmax of the tumor MAT^{LT} and those of the liver MAT^{L} determined in the presence and absence of Dl. Kₘ is µM methionine and Vmax is pmole AdoMet formed per min per enzyme from cells with 1 µg DNA.

Table 5

Helping role of DHI to Boost the Activity of DI

| Additions | Kₘ | Vmax (% of control) | % NBT+ |
|---|---|---|---|
| In vitro: | | | |
| None | 20.8 | 100 | |
| PA, 6.7 mM | 18.5 | 90 | |
| PP-0, 30 µg/ml | 19.2 | 84 | |
| PP-0+PA | 3.3 | 39 | |
| In cell cuture: | | | |
| None | 20.8 | 100 | 3 |
| PA, 4 mM | 20.3 | 88 | 7 |
| PP-0, 20 µg/ml | 17.5 | 64 | 17 |
| PP-0+PA | 2.8 | 20 | 52 |
| RA, 0.15 µM | 17.5 | 60 | 16 |
| RA+PA | 2.9 | 18 | 60 |
| DMFA, 0.3% | 16.4 | 78 | 14 |
| DMFA+PA | 3.0 | 37 | 53 |

(Note: PA is phenylacetate, an inhibitor of MAT. RA is retinoic acid and DMFA is dimethylformamide. Both are Dls mediated through receptors to produce oligoisoadenylate as the Dl. Kₘ is µM of methionine and Vmax is pmole AdoMet formed per minute per enzyme from cells with 1 µg DNA.)

FIG. 5 shows sensitivity of CSCs subpopulation toward cytotoxic drugs and CDA-2. Huh 7 hepatoma cells were incubated with cytotoxic drugs or CDA-2 and the purified active preparation SACN as described. The cell viability was determined by the sulforhodamine B binding assay by flowcytometer.

FIG. 6 shows induction of TD by CDA-2. Hepatoma Smmu 7721 was inoculated to nude mice subcutaneously and let the tumor to grow to the size around 1 cm diameter to start treatment by ip injection of 1 g/kg of CDA-2. The treatment continued for 4 weeks. Then, the tumors were excised for the examination of tissue morphology. Non treated tissue shows disoriented structure on the left, whereas treated tissue shows well organized structure like that of normal liver structure.

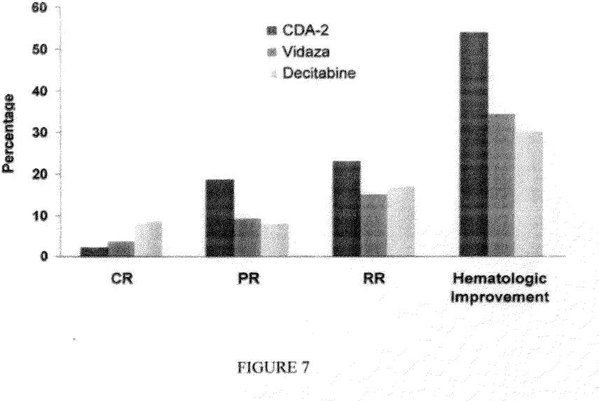

FIG. 7 shows therapeutic efficacy of CDA-2 on MDS, the response rate of CDA-2 in comparison to Vidaza and Decitabine. 117 MDS patients received two cycles of CDA-2 treatment. Each cycle consists of 14 days with daily injection of 200 ml of CDA-2. Vidaza and Decitabine treatments were also two cycle treatments of 28 days. If the treatment is allowed to continue for a longer period, more patients may get into the category of CR. The higher hematological improvement rate of the patients treated with CDA-2 may be an indication that CDA-2 is less toxic.

Table 6

Effective Dls From Our Studies.

| Dls | ED₂₅ | ED₅₀ | ED₇₅ |
|---|---|---|---|
| TPA, nM | 0.17 | 0.26 | 0.36 |
| ATRA, µM | 0.18 | 0.36 | 0.75 |
| PGJ2, µM | 7.9 | 13.8 | 20.5 |
| 16, 16-dimethylPGE2, µM | 10.8 | 17.3 | 30.1 |
| PGE2, µM | 20.6 | 32.0 | 46.5 |
| BicycloPGE2, µM | 21.0 | 43.5 | |
| AA, µM | 24.0 | 46.8 | |
| BIBR1532, µM | 32.3 | 43.7 | 55.1 |
| Boldine, µM | 60.1 | 78.8 | 94.2 |

(Note: Tetradecanoyl phorbol acetate (TPA) and all-trans retinoic acid (ATRA) were well known Dls. PG derivatives were wound healing. metabolites found by us to display impressive Dl activity. Inhibitors of telomerase such as BIBR 1532 and boldine were also our discovery as active Dls.)

Table 7

Effective DHIs of the Present Invention

| SAHH Inhibitors | Rlₒ.₅, µM | Growth Inhibitors | Rl_{0.5,} µM |
|---|---|---|---|
| Pyrvinium pamoate | 0.012 | Signal transduction: | |
| Vitamin D₃ | 0.61 | Sutent | 0.28 |
| Dexamethasone | 0.75 | Berberine | 1.62 |
| Testosterone | 1.55 | Votrient | 10.1 |
| Gugulsterone | 1.59 | Gleevec | 11.9 |
| ß-sitoisterol | 1.72 | Metformin | 44.9 |
| Dihydroepiandrosterone | 1.79 | Non-Specific: | |
| Dihydrotestosterone | 2.10 | AS₂O₃ | 0.28 |
| Prenisolone | 2.22 | CoCl₂ | 0.62 |
| Estradiol | 2.45 | Selenite | 19.7 |
| Progesterone | 3.55 | Polyphenols: | |
| Hydrocortisone | 4.59 | Tannic acid | 0.37 |
| Pregnenolone | 7.16 | Epigallocatechin gallate | 0.62 |
| Pregnenolone sulfate | 7.35 | Resveratrol | 1.16 |
| MT Inhibitors: | | Curcumin | 1.24 |
| Ethidium bromide | 1.10 | Kuromanin | 1.43 |
| Uroerythrin | 1.75 | Coumestrol | 1.95 |
| Hycanthone | 2.10 | Genisteine | 2.16 |
| Riboflavin | 2.90 | Pterostilbene | 2.19 |
| MAT Inhibitors: | | Pyrogallol | 3.18 |
| Indol acetic acid | 220 | Silibinin | 3.80 |
| Phenylacetylvaline | 500 | Caffeic acid | 3.87 |
| Phenylacetylleucine | 780 | Ellagic acid | 4.45 |
| Phenylacetylisoleucine | 800 | Gallic acid | 5.35 |
| Butyric acid | 850 | Ferulic acid | 7.41 |
| Phenylbutyric acid | 970 | Phloroglucinol | 38.8 |

(Note: All DHIs are disclosed for the current invention. The potency Rl₀.₅ was determined as described previously [67]. RI stands for reductive index. Rl_{0.5} is equivalent to ED₂₅ of DI.)

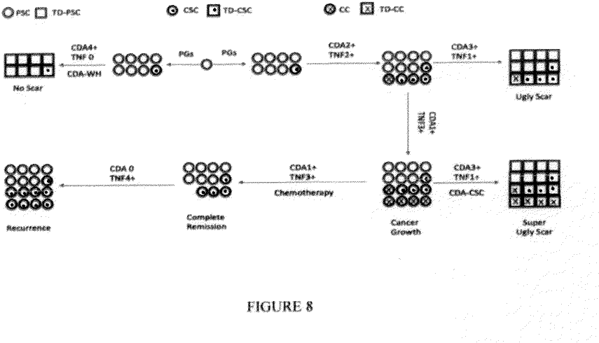

Wound triggers biological response to produce PGs, which are good for the wound healing by promoting the proliferation of PSCs. FIG. 8 shows the relationship of wound to the development of cancer. The proliferation of PSCs always runs a risk for PSCs to evolve into CSCs. Wound healing requires the TD of PSCs mediated through CDA, the natural wound healing metabolites active as Dls and DHls. Healthy persons have enough of CDA components to ensure perfect wound healing. The application of CDA-WH, which is an ointment containing 50% of AA-pregnenolone (1:1), is an added insurance to make no scar wound healing. Wound or sickness also triggers immunological response to cause cachexia symptom resulting in the loss of CDA via excessive urinary excretion. The loss of CDA is bad for wound healing. Whereas PSCs obey the rule of contact inhibition, CSCs may pile up if there is not enough CDA to control their proliferation, resulting in ugly scar if they are eventually induced to undergo TD. The continuous loss of CDA due to the progress of sickness allow cancer cell (CC) to keep on dividing, that requires therapy. CDA-CSC is a preparation of AA + BIBR1532 + pregnenolone + curcumin that can direct TD of CCs,. CSCs, and PSCs to stop the progression of cancer growth. The residual tumor mass becomes super ugly scar. Chemotherapy can take out faster growing CCs alright, which constitute the majority tumor mass. But the inability to take out CSCs and PSCs which are protected by drug resistant mechanism and its contribution to damage CDA laid the ground for inevitable recurrence and fatality.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

### REFERENCES

1. Liau MC, Szopa M, Burzynski B, Burzynski SR. Chemo-surveillance: A novel concept of the natural defense mechanism against cancer. Drug Exptl Clin Res 13(Suppl. 1): 77-82, 1987.

2. Liau MC, Zhuang P, Chiou GCY. identification of the tumor factor of abnormal methylation enzymes as the catalytic subunit of telomerase. Chin Oncol Cancer Res 7: 86-96, 2010.

3. Liau MC, Lee SS, Burzynski SR. Hypomethylation of nuclei acids: A key to the induction of terminal differentiation. Intl J Exptl Clin Chemother 2: 187-199, 1989.

4. Liau MC, Lee SS, Burzynski SR. Modulation of cancer methylation complex isozymes as a decisive factor in the induction of terminal differentiation mediated by Antineoplaston A5. Intl J Tiss React 12(Suppl.): 27-36, 1990.

5. Liau MC, Chang CF, Saunders GS, Tsai YH. S-Adenosylhomocysteine hydrolases as the primary target enzymes in androgen regulation of methylation complexes. Arch Biochem Biophys 208(1): 261-272, 1981.

6. Liau MC, Baker LL. Destruction promotes the proliferation of progenitor stem cells and cancer stem cells. Therefore, non-destruction is a better strategy for cancer therapy: A commentary. J Pharmacol Pharmaceu Pharmacovigi 4: 029, 2021. DOI: 10.24966/PPP5649/100029.

7. Liau MC, Baker LL. The proliferation and the terminal differentiation of progenitor stem cells determine the success of wound healing. Likewise, the proliferation and the terminal differentiation of cancer stem cells determine the success of cancer therapy. Nov Res Sci 6(3): NRS. 000636, 2021.

8. Hwa J, Martin K. Chapter 18: The eicosanoids: prostaglandins, thromboxanes, leucotrienes, and related compounds. In: Katzung BG (ed.) Basic and Clinical Pharmacology (14^{th} ed.) New York, NY: McGraw-Hill Education, 2017.

9. Ho ATV, Palla AR, Blake MR, Yual ND, et al. Prostaglandin E2 is essential for efficacious skeletal muscle stem cells function, augmenting regeneration and strength. Proc Natl Acad Sci USA 114(26): 6675-6684, 2017.

10. Liau MC, Kim JH, Fruehauf JP. In pursuance of differentiation inducers to combat cancer via targeting of abnormal methylation enzymes. J Cancer Tumor Intl 10(2): 39-47, 2020.

11. Liau MC, Kim JH, Fruehauf JP. Arachidonic acid and its metabolites as the surveillance differentiation inducers to protect healthy people from becoming cancer patients. Clin Pharmacol Toxicol Res 4(1): 7-10, 2021.

12. Riciotti E, FitzGerald GA. Prostaglandin and inflammation. Arteroscher Thromb Vase Biol 31(5): 986-1000, 2011.

13. Liau MC, Baker LL. The functionality of chemo-surveillance dictates the success of wound healing as well as cancer therapy. Nov Res Sci 7(2): NRS. 000657, 2021.

14. Muller T, Gessi M, Waha A, Isselstein U, et al. Nuclear exclusion of TET-1 is associated with loss of 5-hydroxymethylcytosine in IDH1 wild type glioma. Am J Pathol 181(2):675-683, 2012.

15. Yang H, Lin Y, Bai F, Zhang JY, et al. Tumor development is associated with decrease of TET gene expression and 5-methylcytosine hydroxylation. Oncogene 32(5): 663-669, 2013.

16. Kudo Y, Tateishi K, Yamamoto K, Yamamoto S, et al. Loss of 5-hydroxymethylcytosine is accompanied with malignant cellular transformation. Cancer Sci 103(4): 670-676, 2012.

17. Ficz G, Gribben JG. Loss of 5-hydrozymethylcytosine in cancer: cause or consequence? Genomics 104(5): 352-357, 2014.

18. Lin J, Jiang J, Mu J, Lin D, et al. Global DNA 5-hydroxymethylcytosine and 5-formylcytosine contents are decreased in the early stage of hepatocellular carcinoma. Hepatology 69(1): 196-208, 2019.

19. Williamson PJ, Kruger AR, Reynolds PJ, Hambin TJ, et al. Establishing the incidence of myelodysplastic syndromes. Br J Haemato 87(4): 743-745, 1994.

20. Boula A, Vougarelis SM, Grennouli S, Kotrinakis G, et al. Effect of cA2 antitumor necrosis factor-a antibody therapy on hematopoiesis of patients with myelodysplastic syndromes. Clin Cancer Res 12(10): 3099-3108, 2008.

21. Itkin T, Rafii S. Leukemia cells “gas up” leaky bone marrow blood vessels. Cancer Cell 32(3): 276-278, 2017.

22. Passaro D, Di Tullio A, Abarrategi A, Ronault-Pierre K, et al. Increased vascular permeability in the bone marrow microenvironment contributes to disease progression and drug response in acute myeloid leukemia. Cancer Cell 32(3): 324-341, 2017.

23. Counter CM, Gupta J, Harley CB, Leber B, et al. Telomerase activity in normal leukocytes and hematological malignancies. Blood 85(9): 2315-2320, 1995.

24. Fu C, Chen Z. Telomerase activity in myelodysplastic syndrome. Chin Med J (Eng) 115(10): 1075-1078, 2002.

25. Larson CA, Cote G, Quintas-Cardama A. The changing mutational landscape of acute myeloid leukemia and myelodysplastic syndrome. Mol Cancer Res 11(8): 815-827, 2013.

26. Papaemmanuil E, Gerstung M, Malconvati L, Tauro S, et al. Clinical and biological implication of driver mutations in myelodysplastic syndromes. Blood 124(17): 2705-2712, 2013.

27. Kennedy JA, Ebert BL. Clinical implication of genetic mutations in myelodysplastic syndrome. J Clin Oncol 35(9): 967-974, 2017.

28. Well PS, Kjallquist U, Chowdhury O, Doolittle H, et al. Myelodysplastic syndromes are propagated by rare and distinct human cancer stem cells in vivo. Cancer Cell 25(6): 794-808, 2014.

29. Maeck L, Hasse D, Schoch D, Hiddemann W, et al. Genetic instabilithy in myelodysplastic syndrome: Detection of microsatellite instability and loss of heterozygosity in bone marrow sample with karyotype alteration. Br J Haemato 109(4): 842-846, 2000.

30. Xie D, Hefmann KW, Mori N, Miller CW. Allotype analysis of the myelodysplastic syndrome. Leukemia 14: 805-810, 2001.

31. Kuramoto K, Ban S, Oda K, Tanaka H, et al. Chromosomal instability and radiosensitivity in myelodysplastic syndrome. Leukemia 16(10): 2253-2258, 2002.

32. Takada A, Goosby C, Yaseen NR. NUP98-HOXA 9 induces long term proliferation and blocks differentiation of primary human CD34+ hematopoietic cells. Cancer Res 66(13): 6628-6637, 2006.

33. Kampalath BN, Liau MC, Burzynski B, Burzynski SR. Chemoprevention by Antineoplaston A10 of benzopyrene induced pulmonary neoplasia. Drug Exptl Clin Res 13(Supp..1): 51-56, 1987.

34. Kampalath BN, Liau MC, Burzynski B, Burzynski SR. Protective effect of Antineoplaston A10 in hepatocarcinogenesis induced by alphatoxin B1. Intl J Tissue React 12(Suppl.): 43-50, 1990.

35. Liau MC, Chang CF, Becker FF. Alteration of S-adenosylmethionine synthetases during chemical hepatocarcinogenesis and in resulting carcinomas. Cancer Res 39: 2113-2119, 1979.

36. Liau MC, Chang CF, Giovanella BC. Demonstratiopn of an altered S-adenosylmethionine synthetase in human malignant tumors xenografted into athymic nude mice. J Natl Cancer Inst 64 (5): 1071-1075, 1980.

37. Liau MC, Fruehauf JP. Restoration of the chemo-surveillance capability is essential for the success of chemotherapy and radiotherapy to put cancer away. Adv Complement Alt Med 5(4): ACAM.000617, 2019.

38. Zhou S, Schuetz JD, Bunting KD, Colapietro AM, et al. The ABC transporter Bcrp1/ABCG2 is expressed in a wide variety of stem cells and is a molecular determinant of theside population phenotype.Nat Med 7: 1028-1034, 2001.

39. Moitra K, Lou H, Dean M. Multidrug efflux pumps and cancer stem cell in sights into multidrug resistance and therapeutic development. Clin Pharmacol Ther 89: 491-502, 2011.

40. Frome FM, Maitland NJ. Cancer stem cell, model of study and implication of therapy resistant mechanism. Adv Exp Med Biol 720: 105-118, 2011.

41. Zhang M, Atkinson Rl, Rosen M. Selective targeting of radiation resistant tumor initiating cells. Proc Natl Acad Sci USA 107: 3522-3527, 2010.

42. Jordan CT, Guzman ML, Noble M. Cancer stem cells. N Eng J Med 355(12): 1253-1261, 2006.

43. Zabierowski SE, Herlyn M. Melanoma stem cells: The dark seed of melanoma. J Clin Oncol 26(17): 2890-2894, 2008.

44. Hemmings C. The elaboration of a critical framework for understanding cancer: The cancer stem cell hypothesis. Pathology 42(2): 105-112, 2010.

45. Liau MC, Fruehauf JP. It has been half a century since President Nixon declared war on cancer: Destabilization of abnormal methylation enzymes has the blessing of the nature to win the war on cancer. Adv Complement Alt Med 6(1): ACAM.000630, 2020.

46. Liau MC, Baker LL. Wound healing, evolution of cancer and war on cancer. Intl Res J Oncol 4(3): 13-20, 2021.

47. Liau MC, Fruehauf JP. The winner of the contest to eradicate cancer stem cells wins the contest of cancer therapies: The winner is cell differentiation agent formulations. Adv Complement Alt Med 5(4): ACAM.000620, 2020.

48. Liau MC. Pharmaceutical composition inducing cancer cell differentiation and the use for the treatment and prevention of cancer thereof. U.S. Pat. 7232578 B2, 2007.

49. Ma J. Differentiation therapy of malignant tumor and leukemia. CSCO Treaties on the Education of Clinical Oncology: 480-486, 2007.

50. Liau MC, Fruehauf JP. Destabilization of abnormal methylation enzymes as a critical mechanism for CDA-2 to reverse MDS progression. The First International Forum of Myelodysplastic Syndrome in Tai Zhou, Jiangsu, China: 31-35, 2015.

51. Prassana P, Shack S, Wilson VL, Samid D. Phenylacetate in chemoprevention of 5-aza-2'-deoxycytidine-induced carcinogenesis. Clin Cancer Res 1(18): 865-871, 1995.

52. Palii SS, van Emburgh BO, Sankpal UT, , Brown KD, et al. DNA methylation inhibitor 5-aza-2'-deoxycytidine induces reversible DNA damage that is distinctly influenced by DNA methyltransferase 1 and 3B. Mol Cell Biol 28(2): 752-771, 2008.

53. Kizietepe T, Hideshima T, Catley L, Raje N. 5-Azacytidine, a DNA methyltransferase inhibitot, induces ATR-mediated DNA-double strand break responses, apoptosis, and synergistic cytotoxicity with doxorubicin and bortezomib against multiple myeloma cells. Mol Cancer Ther 6(6): 1718-1727, 2007.

54. Yang Q, Wu F, Wang F, Cai K, et al. Impact of DNA methyltransferase inhibitor 5-azacytidine on cardiac development of zebrafish in vivo and cardiomyocyte proliferation, apoptosis, and the homeostasis of gene expression in vitro. J Cell Biochem 120(10): 17459-17471, 2019.

55. Liau MC, Baker LL. Abnormal methylation enzymes as the bullseye of targeted cancer therapies. Nov Res Sci 7(4): NRS.000667, 2021.

56. Racanelli AC, Turner FB, Xie LY, Moran RG. A mouse gene that coordinates epigenetic controls and transcriptional interference to achieve tissue specific expression. Mol Cell Biol 28(2):836-848, 2008.

57. Liau MC, Hunt ME, Hurlbert RB.. Role of ribosomal RNA methylases in the regulation of ribosome production. Biochemistry 15(14): 3158-3164, 1976.

58. Bernstein KA, Bleichert F, Bean JM, Cross FR, et al. Ribosome biogenesis is sensed at the start of cell cycle checkpoint. Mol Biol Cell 18(3): 953-964, 2007.

59. Justilien V, Ali SA, Jamieson 1, Yin N, et al. Ect₂-dependent rRNA synthesis is required for KRAS-TRP₅₃-driven lung adenocarcinoma. Cancer Cell 31(2): 256-269, 2017.

60. Liau MC. Abnormal methylation enzymes: A selective molecular target for differentiation therapy of cancer. Chin Pharm J 56(2): 57-67, 2004.

61. Prudova A, Bauman Z, Braun A, Vitvitsky V, et al. S-Adenosylmethionine stabilizes cystathionine β-synthase and modulate redox capacity. Proc Natl Acad Sci USA 103(17): 6489-6494, 2006.

62. Chiba P, Wallner C, Kaizer E. S-Adenosylmethionine metabolism in HL-60 cells: Effect of cell cycle and differentiation. Biochim Biophy Acta 971(1): 38-45, 1988.

63. Burzynski SR, Georgiades J. Effects of urinary peptides on DNA, RNA, and protein synthesis in normal and neoplastic cells. Fed Proc 323: 766, 1973.

64. Burzynski SR, Loo TL, Ho DH, Rao PN, et al. Biologically active peptides in human urine: III. Inhibitors of the growth of human leukemia, osteosarcoma and Hela cells. Physiol Chem Phys 8: 13, 1976.

65. Liau MC, Fruehauf PA, Zheng ZH, Fruehauf JP. Development of synthetic CDA formulations for the prevention and therapy of cancer via targeting of cancer stem cells. J Cancer Studies Ther 4(1): 1-19, 2019.

66. Liau MC, Liau CP, Burzynski SR. Potentiation of induced terminal differentiation by phenylacetic acid and related chemicals. Intl J Exptl Clin Chemother 5(1): 9-17, 1992.

67. Liau MC, Huang U, Lee JH, Chen SC, et al. Development of differentiation helper inducer for the differentiation therapy of cancer. Chin Pharm J 50: 289-303, 1998.

68. Liau MC, Liau CP. Methyltransferase inhibitors as excellent differentiation helper inducers for differentiation therapy of cancer. Bull Chin Cancer 11(3):166-168, 2002.

69. Liau MC, Kim JH, Fruehauf JP. Potentiation of ATRA activity in HL-60 cells by targeting methylation enzymes. J Pharmacol Pharmaceu Pharmacovigi 3(1): 9-17, 2019

70. Nice J, Liu L, Jones PA. Variable effects of DNA synthesis inhibitors upon DNA methylation in mammalian cells. Nucleic Acid Res 14(10): 4353-4367, 1986.

71. Nice J. Drug induced DNA hypermethylation and drug resistance in human tumors. Cancer Res 49(21): 5829-5836, 1989.

72. Liau MC, Luong Y, Liau CP, Burzynski SR. Prevention of drug induced DNA hypermethylation by antineoplaston components. Intl J Exptl Clin Chemother 5(1); 19-27, 1992.

73. Liau MC, Fruehauf JP. Think hard on the destabilization of abnormal methylation enzymes to combat cancer: It can be life saving. Adv Complement Alt Med 5(3): ACAM.000615, 2019.

74. Liau MC, Kim JH, Fruehauf JP. Destabilization of abnormal methylation enzymes to combat cancer: The nature’s choice to win the war on cancer. Lambert Academic Publishing 978-620-2-66889-7, 2020.

75. Mougeri-Sacca M, Dimarino S, De Maria R. Biological and clinical implication of cancer stem cells in primary brain tumors. Front Oncol 3:6, 2013.

76. Burzynski SR, Nagy-Kubove E. Treatment of esthesion neuroblastoma and non-small cell-lung cancer with phenylbutyrate. J Cancer Ther 2(4): 518-522, 2011.

77. Burzynski SR, Tamicki TJ, Burzynski GS, Brookman S. Preliminary findings on the use of targeted therapy with pazopanib and other agents in combination with sodium phenybutyrate in the treatment of glioblastoma multiforme. 1 Cancer Ther 5(14): 1423-1437, 2014.

78. Burzynski SR, Burzynski GS, Brookman S. A case of sustained objective response of recurrent/progressive diffuse intrinsic pontine glioma with phenylbutyrate and targeted agents. 1 Cancer Ther 6(1): 40-44, 2015.

79. Baker MJ, Brem S, Daniels S, Sherman B, et al. Complete response of a recurrent, multicentric malignant glioma in a patient treated with phenylbutyrate. J Neuroonco 59(3): 239-242, 2002.

80. Fruehauf JP, Liau MC. Therapeutic targeting of HIF to reverse the cancer stem phenotype and epithelial-mesenchymal transition. Therapy 8:737-740, 2011.

What is claimed is:

1. A method for administration of an active ingredient to a subject to destabilize abnormal methylation enzymes for perfection of wound healing, comprising: administering to the subject an effective amount of a pharmaceutical composition comprising: a differentiation inducer, a differentiation helper inducer, or an anti-cachexia chemical as the active ingredient.

2. The method of claim 1, wherein the subject is human.

3. The method of claim 1, wherein the differentiation inducer is selected from a group consisting of prostaglandin J2, 16,16-dimethylprostaglandin E2, prostaglandin E2,. bicycloprostaglandin E2, arachidonic acid, BIBR1532, and boldine.

4. The method of claim 1, wherein the differentiation helper inducer is an SAHH inhibitor selected from a group consisting of pyvinium pamoate, vitamin D₃, dexamethasone, testosterone, gugusterone, B-sitosterol, dehydroepiandrosterone, dihydrotestosterone, prednisolone, estradiol, progesterone, hydrocortisone, pregnenolone, and pregnenolone sulfate.

5. The method of claim 1, wherein the differentiation helper inducer is an MT inhibitor selected from a group consisting of ethidium bromide, uroerythrin, hycanthone, and riboflavin.

6. The method of claim 1, wherein the differentiation helper inducer is an MAT inhibitor selected from a group consisting of indolacetic acid, phenylacetylvaline, phenylacetylleucine, phenylacetylisoleucine, butyric acid, and phenylbutyric acid.

7. The method of claim 1, wherein the differentiation helper inducer is a polyphenol selected from a group consisting of tannic acid, epigallocatechin gallate, resveratrol, curcumin, kuromanin, coumestrol, genisteine, pterostilbene, pyrogallol, silibinin, caffeic acid, ellagic acid, gallic acid, ferulic acid, and phloroglucinaol.

8. The method of claim 1, wherein the differentiation helper inducer is a signal transduction and growth inhibitor selected from a group consisting of sutent, berberine, vorient, gleevec, metformin, As₂O₃, CoCl₂, and selenite.

9. The method of claim 1, wherein the anti-cachexia chemical is phenylacetylglutamine.

10. The method of claim 1, wherein said perfection of wound healing comprises an indication of prevention of scar or treatment of cancer.

11. The method of claim 1, wherein said pharmaceutical composition has a form suitable to be administered orally, parenterally, or topically to said subject.

12. The method of claim 10, wherein said pharmaceutical composition comprises CDA-WH, which is an ointment preparation suitable for topical administration for the prevention of scar, and wherein said preparation is 50% arachidonic acid:pregnenolone (1:1) in an ointment base.

13. The method of claim 10, wherein said pharmaceutical composition comprises CDA-CSC, which is a parenteral preparation suitable for the treatment of cancer to direct terminal differentiation (TD) of cancer stem cells (CSCs) and cancer cells (CC), and wherein said preparation is a mixture of 36 mg of arachidonic acid +52 mg of BIBR1532 + 22.5 mg of pregnenolone + 2.3 mg of curcumin in 5 ml of saline solution as a single dose.

14. The method of claim 13, wherein said pharmaceutical composition of CDA-CSC further comprises 10 g phenylacetylglutamine capsules.

15. The method of claim 1, wherein said perfection of wound healing comprises an indication of treatment of myelodysplastic syndrome (MDS).

16. The method of claim 15, wherein said pharmaceutical composition comprises CDA-MDS, which is a parenteral preparation suitable for the treatment of MDS, wherein the preparation is a mixture of 70.5 mg of arachidonic acid + 34 mg of pregnenolone in 5 ml of saline solution as a single dose.

17. The method of claim 16, wherein said pharmaceutical composition of CDA-MDS further comprises 10 g phenylacetylglutamine capsules.

18. The method of claim 1, wherein said perfection of wound healing comprises an indication of treatment of malignant brain tumor.

19. The method of claim 18, wherein said pharmaceutical composition comprises CDA-BT, which is a parenteral preparation suitable for the treatment of malignant brain tumor, wherein the preparation is a mixture of 70.5 mg arachidonic acid + 22.5 mg of pregnenolone + 0.3 mg of pyrvinium pamoate in 5 ml of saline solution as a single dose.

20. The method of claim 1, wherein said perfection of wound healing comprises an indication of treatment of melanoma, and wherein said pharmaceutical composition comprises CDA-Mel, which is a parenteral preparation suitable for the treatment of melanoma, wherein the preparation is a mixture of 36 mg of arachidonic acid + 52 mg of BIBR1532 + 52.5 mg of resveratrol in 5 ml of saline solution as a single dose.
